(19) United States
(12) Patent Application Publication
(10) Pub. No.: US 2014/0194726 A1
(43) Pub. Date: Jul. 10, 2014

(54) Ultrasound Neuromodulation for Cognitive Enhancement

(71) Applicants: David J. Mishelevich, (US); Neurotrek, Inc., Los Gatos, CA (US)

(72) Inventors: David J. MISHELEVICH, Playa del Rey, CA (US); Daniel Z. WETMORE, San Francisco, CA (US); William J. TYLER, Roanoke, VA (US); Tomo SATO, Roanoke, VA (US)

(73) Assignee: Neurotrek, Inc., Los Gatos, CA (US)

(21) Appl. No.: 13/734,216

(22) Filed: Jan. 4, 2013

(51) Int. Cl. A61N 7/00; A61B 6/03; A61B 5/0482; A61B 5/055

(52) U.S. Cl. CPC A61N 7/00; A61B 5/055; A61B 6/037; A61B 5/0482; A61N 2007/0026
USPC 600/411; 600/427; 600/545; 601/2

(57) ABSTRACT

Disclosed are methods and systems for non-invasive neuromodulation using ultrasound for cognitive enhancement. Cognitive enhancement can be used for mitigation of abnormal conditions such as Alzheimer's Disease, Parkinson's Disease or stroke, or for enhancement in a normal individual. The neuromodulation can produce acute or long-term effects. The latter occur through Long-Term Depression (LTD) and Long-Term Potentiation (LTP) via training. Included is control of direction of the energy emission, intensity, frequency, pulse duration, pulse pattern, mechanical perturbation, and phase/intensity relationships to targeting and accomplishing up regulation and/or down regulation.

### CROSS REFERENCE TO RELATED APPLICATIONS

This non-provisional patent application is a continuation in part of U.S. patent application Ser. No. 12/940,052, filed on Nov. 5, 2010, entitled “NEUROMODULATION OF DEEP-BRAIN TARGETS USING FOCUSED ULTRASOUND”; which application claims priority to U.S. Pat. App. Ser. No. 61/260,172, filed on Nov. 11, 2009; which application claims priority to U.S. Pat. App. Ser. No. 61/295,757, filed on Jan. 17, 2010; and claims priority to U.S. Pat. App. Ser. No. 61/583,199, entitled “ULTRASOUND NEUROMODULATION FOR COGNITIVE ENHANCEMENT,” filed Jan. 5, 2012; the entire disclosures of which are incorporated herein by reference.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity for improvement of function.

### BACKGROUND

Ultrasound (US) has been used for many medical applications, and is generally known as cyclic sound pressure with a frequency greater than the upper limit of human hearing. The production of ultrasound is used in many different fields, typically to penetrate a medium and measure the reflection signature or to supply focused energy. For example, the reflection signature can reveal details about the inner structure of the medium. A well-known application of this technique is its use in sonography to produce a picture of a fetus in a womb. There are other applications which may provide therapeutic effects, such as lithotripsy for ablation of kidney stones or high-intensity focused ultrasound for thermal ablation of brain tumors. An important benefit of ultrasound therapy is its non-invasive nature. US waveforms can be defined by their acoustic frequency, intensity, waveform duration, and other parameters that vary the timecourse of acoustic waves in a target tissue. US waveforms based on pulses less than about 1 second are generally referred to as pulsed ultrasound and are repeated at a rate equivalent to the pulse repetition frequency. Tone bursts that extend for about 1 second or longer—though, strictly speaking, also pulses—are often referred to as continuous wave (CW).

Ultrasound can be defined as low or high intensity. In contrast to transcranial ultrasound neuromodulation, ultrasound imaging generally employs high intensity (greater than about 1 W/cm²), high frequency ultrasound (greater than about 1 MHz). In ultrasound, acoustic intensity is a measure of power per unit of cross sectional area (e.g. mW/cm²) and requires averaging across space and time. The intensity of the acoustic beam can be quantified by several metrics that differ in the method for spatial and temporal averaging. These metrics are defined according to technical standards established by the American Institute for Ultrasound in Medicine and National Electronics Manufacturers Administration (NEMA. Acoustic Output Measurement Standard For Diagnostic Ultrasound Equipment (National Electrical Manufacturers Association, 2004)). A commonly used intensity index is the ‘spatial-peak, temporal-average’ intensity (Iₛₚₜₐ), and the Iₛₚₜₐ can be defined as the maximum intensity in the beam averaged over the pulse repetition period. The Iₛₚₜₐ can be related to the amount of heat delivered to a tissue by ultrasound.

Although it has been demonstrated that focused ultrasound directed at neural structures can stimulate those structures, the prior methods and apparatus have lead to less than ideal results in at least some instances. If neural activity is increased or excited, the neural structure is up regulated; if neural activated is decreased or inhibited, the neural structure is down regulated. Neural structures are usually assembled in circuits. For example, nuclei and tracts connecting them make up a circuit.

The effect of ultrasound on neural activity appears to be at least two fold. Firstly, increasing temperature will increase neural activity. Secondly, mechanical perturbation appears to be related to the opening of ion channels related to neural activity. With regards to increasing temperature, an increase up to 42 degrees C. (say in the range of 39 to 42 degrees C.) locally for short time periods will increase neural activity in a way that one can do so repeatedly and be safe. For clinical uses, it can be helpful to ensure that the temperature does not rise about 50 degrees C. or tissue will be destroyed (e.g., 56 degrees C. for one second). For example, another therapeutic application of ultrasound is to ablate tissue so as to permanently destroy the tissue (e.g., for the treatment of cancer). An example is the ExAblate device from InSightec in Haifa, Israel.

With regard to mechanical perturbation, an explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm2 upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, William, James P., PCT/US2009/050560, WO 2010/009141, published 2011 Jan. 21). Tyler incorporated this approach in two patent applications he submitted (Tyler, William, James P., PCT/US2009/050560, WO 2010/009141, “Methods and Devices for Modulating Cellular Activity Using Ultrasound,” published 2011 Jan. 21 and “Devices and Methods for Modulating Brain Activity,” PCT/US2010/055527, WO 2011/057028, published 2011 May 12). Alternative mechanisms for the effects of ultrasound may be discovered as well. In fact, multiple mechanisms may come into play.

Several potential mechanisms for the conversion of mechanical energy into neuronal activity have been proposed. Neurons are mechanically sensitive and can act as a piezoelectric material by converting a mechanical displacement into electrical currents or membrane polarization. Stretch-induced activation or inactivation of ion channels is one mechanism for converting mechanical force into currents that modulate neuronal activity. An additional or alternative mechanism of stretch-induced effects in ion channels may relate to mechanical effects on cytoskeletal proteins such as actin or tubulin that could then be transduced to membrane-bound ion channels through the cytoskeletal structure.

Flexoelectric effects are another mechanism for converting mechanical energy into changes in neuronal activity. Thermodynamic investigations of lipid-phase transitions have shown that mechanical waves can be adiabatically propagated through lipid monolayers and bilayers, as well as neuronal membranes to influence fluidity and excitability. Notably, such sound wave propagation in pure lipid membranes has been estimated to produce depolarizing potentials ranging from 1 to 50 mV with negligible heat generation (˜0.01 K), potentially via a flexoelectric effect. In this manner, mechanical energy delivered by an acoustic wave can cause membrane polarization and affect voltage-gated channels and thus neuronal activity.

Another potential mechanism for neuromodulation by ultrasound is by causing changes in blood flow through mechanical and/or thermal effects.

Some people have questioned the ethics of using means to enhance cognitive function in a person with normal cognition. (Mendelsohn, D. Lipsman, N. and M. Bernstein, “Neurosurgeons' Perspectives on Psychosurgery and Neuroenhancement: a Qualitative Study at One Center,” J. Neurosurg. 2010 December; 113(6):1212-8. Epub 2020 June 4), and it would be helpful to provide methods and apparatus that can enhance cognitive function in a person with normal cognition in a safe and effective manner.

The prior methods and apparatus of treating cognitive function can provide less than ideal results in at least some instances. For example, electrical stimulation is limited in focusing and may require implanted electrodes in many instances and can rely on invasive and potentially dangerous implantation surgery in at least some instances. Although prior methods and apparatus have demonstrated that focused ultrasound directed at neural structures can affect neural activity, the prior methods and apparatus can be less than ideally suited to improve cognitive function. For example, the prior waveforms and targeted neural structures may provide less than ideal cognitive improvement when applied in amounts below the damage threshold in at least some instances. Also, the frequencies, intensities, and pulse durations of the prior methods and apparatus can be less than ideal for treating cognitive function. Further, the stimulation of neural structures with ultrasound can be somewhat unpredictable, and the stimulation of neural structures with prior ultrasound methods and apparatus can provide results that are less predictable than would be ideal in at least some instances.

Because of the deficiencies of the prior methods and apparatus to enhance cognitive function, it would be beneficial to provide improved methods and apparatus of enhancing cognitive function.

### SUMMARY

Embodiments of the present invention provide improved methods and systems for non-invasive neuromodulation using ultrasound for cognitive enhancement, which can be based on the neuromodulation of deep-brain structures in order to enhance cognitive function. Embodiments as described herein provide improved cognitive function with decreased amounts of heating to the targeted neurological structure. Cognitive enhancement can be used for mitigation of abnormal conditions such as stroke, or for enhancement in a normal individual. Such neuromodulation can produce acute effects or Long-Term Potentiation (LTP) or Long-Term Depression (LTD). The methods and apparatus provide control of direction of the energy emission, intensity, frequency (carrier frequency and/or neuromodulation frequency), pulse duration, pulse pattern, and phase/intensity relationships to targeting, so as to provide one or more of increased up-regulation or increased down-regulation with decreased amounts of ultrasound energy to the targeted neural structure. Use of ancillary monitoring or imaging to provide feedback is optional. Embodiments combined with concurrent imaging may comprise non-ferrous material.

One or more targets can be neuromodulated singly or in groups for cognitive enhancement. Cognitive enhancement can be provided for at least two purposes. First, cognitive enhancement can be provided where cognitive faculties have been diminished (e.g., Alzheimer's Disease, Parkinson's disease, Creutzfeld-Jacob disease, Attention Deficit Hyperactivity Disorder, dementia, and stroke). Second, cognitive function in a normal individual can be enhanced, and the normal individual may generally comprise a subject without diminished cognitive function. Thus the type of application of cognitive enhancement can be to abnormal function or normal function, and combinations thereof.

Embodiments may provide a tune up to enhance learning for a student studying for a test, for example so as to concretize learning.

In many embodiments, calendar calculation is used to identify targets for cognitive enhancement.

To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS).

Targets depend on specific patients and relationships among the targets. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets. The ultrasound pulses as described herein can be used in many ways. The pulses can be used at one or more sessions to diagnose the patient, confirm subsequent treatment, or treat the patient, and combinations thereof. The pulses can be shaped in one or more ways, and can be shaped with macro pulse shaping, amplitude modulation of the pulses, and combinations thereof, for example.

In many embodiments, the amplitude modulation frequency of lower than 500 Hz is applied for inhibition of neural activity. The amplitude modulation frequency of lower than 500 Hz can be divided into pulses 0.1 to 20 msec. repeated at frequencies of 2 Hz or lower for down regulation. The amplitude modulation frequency for excitation can be in the range of 500 Hz to 0.25 MHz. The amplitude modulation frequency of 500 Hz or higher may be divided into pulses 0.1 to 20 msec. repeated at frequencies higher than 2 Hz for up regulation.

The targeting, aiming and treatment of the pulsed beam can be done with one or more of known external landmarks, an atlas-based approach or imaging (e.g., fMRI or Positron Emission Tomography), for example. The imaging can be done as a one-time set-up or at each session although not using imaging or using it sparingly is a benefit, both functionally and in terms of the cost of administering the therapy.

While ultrasound can be focused down to a diameter on the order of one to a few millimeters (depending on the frequency), whether such a tight focus is required depends on the conformation of the neural target.

### BRIEF DESCRIPTION OF THE DRAWINGS

FIG. 1 shows ultrasonic-transducer targeting of the Orbito-Frontal Cortex and the Anterior Temporal Lobe for the enhancement of cognitive function, in accordance with embodiments;

FIGS. 2A and 2B illustrate effects of mechanical perturbation of the ultrasound transducer, in accordance with embodiments;

FIG. 3 shows a block diagram of the control circuit, in accordance with embodiments;

FIG. 4 shows transcranial ultrasound neuromodulation waveform and pulsed ultrasound protocol, in accordance with embodiments;

FIG. 5 shows transcranial ultrasound neuromodulation waveform and continuous wave ultrasound protocol, in accordance with embodiments; and

FIG. 6 shows transcranial ultrasound neuromodulation waveform repetition, in accordance with embodiments.

### DETAILED DESCRIPTION

The embodiments as described herein provide methods and systems for neuromodulation of deep-brain targets using ultrasound for cognitive enhancement. Such neuromodulation systems can produce applicable acute or long-term effects. In an embodiment, long-term effects are mediated by long-term depression (LTD) or long-term potentiation (LTP) induced by transcranial ultrasound (US) neuromodulation. Included is control of direction of the energy emission, intensity, frequency (acoustic carrier frequency, amplitude modulation frequency, and/or pulse-repetition frequency), pulse duration, pulse pattern, and phase/intensity relationships to targeting and accomplishing up-regulation and/or down-regulation.

The ultrasound treatment may comprise an ultrasound carrier having a carrier frequency, and the duration and pulse-repetition frequency may be imposed on the ultrasound carrier. For example, the ultrasound carrier frequency can be within a range from 0.3 MHz to 0.8 MHz, and the pulse frequency imposed on the carrier can be within a range from about 10 kHz to about 50 Hz (period of 0.1 to 20 milliseconds, for example).

Transcranial ultrasound neuromodulation (also referred to as bioTU) is a beneficial form of noninvasive brain stimulation for enhancing cognitive function achieved through the modulation of brain circuit activity induced by patterned, local vibration of brain tissue using US having an acoustic frequency greater than about 100 kHz and less than about 10 MHz. Transcranial ultrasound neuromodulation transmits mechanical energy through the skull to its target in the brain without causing significant thermal or mechanical damage and induces neuromodulation. Transcranial ultrasound neuromodulation employs low intensity ultrasound such that the spatial-peak, temporal-average intensity (Iₛₚₜₐ) of the transcranial ultrasound neuromodulation protocol is less than about 1 W/cm² in the targeted brain tissue. The acoustic intensity measure Iₛₚₜₐ is calculated according to established techniques well known to those skilled in the art that relate to the ultrasound acoustic pressure and other transcranial ultrasound neuromodulation protocol characteristics such as the temporal average power during the transcranial ultrasound neuromodulation waveform duration. To provide a large matrix of complex patterns of localized brain tissue vibration, US may be delivered as short-lived continuous waves less than about 5 seconds or in a pulsed manner during transcranial ultrasound neuromodulation protocols such that diverse patterns of neuromodulation can be delivered to achieve communication as herein described. For modulating the activity of brain circuits through localized tissue vibration, transcranial ultrasound neuromodulation protocols may utilize US waveforms of any type known in the art including but not limited to amplitude modulated waveforms, tone-bursts, pulsed waveforms, and continuous waveforms, for example.

In a preferred embodiment, transcranial ultrasound neuromodulation is used to enhance cognitive function in a subject. One or more ultrasound transducers are coupled to the head of an individual subject such as a human or animal (the ‘recipient’).

U.S. patent application Ser. No. 12/940,052, filed on Nov. 5, 2010, entitled “NEUROMODULATION OF DEEP-BRAIN TARGETS USING FOCUSED ULTRASOUND”, U.S. Pub. No. 2011/0112394, in the name of Mishelevich; David J., describes methods and apparatus suitable for combination in accordance with embodiments as described herein.

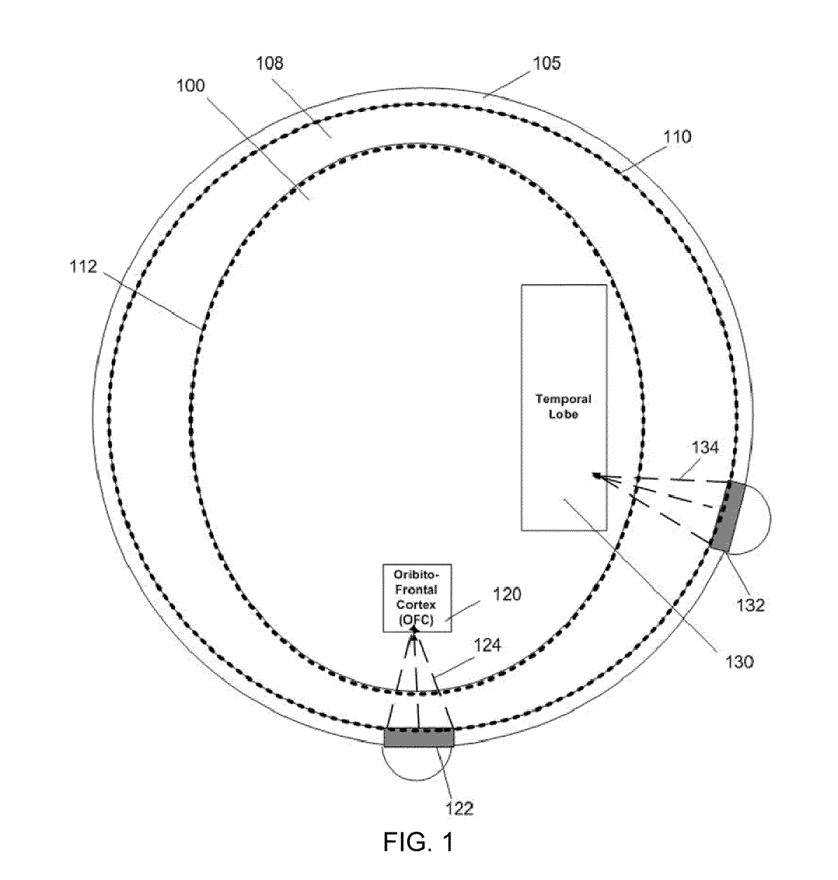

FIG. 1 shows a set of ultrasound transducers targeting for cognitive enhancement. Head 100 contains two targets, Orbito-Frontal Cortex (OFC) 120 and Anterior Temporal Lobe 130. While these two targets are covered here, others can work as well, identified currently or in the future. The targets shown are hit by ultrasound from transducers 122 and 132 fixed to track 105. Ultrasound transducer 122 with its beam 124 is shown targeting the Orbito-Frontal Cortex (OFC) 120 and transducer 132 with its beam 134 is shown targeting Anterior Temporal Lobe 130. For ultrasound to be effectively transmitted to and through the skull and to brain targets, coupling must be put into place. Ultrasound transmission (for example Dermasol from California Medical Innovations) medium 108 is interposed with one mechanical interface to the frame 105 and ultrasound transducers 122 and 132 (completed by a layer of ultrasound transmission gel layer 110) and the other mechanical interface to the head 100 (completed by a layer of ultrasound transmission gel 112). Among other potential targets are the Left Hippocampus, Left Frontal Cortex, Left Middle Temporal Lobe, Ventral Tegmentum, Hypothalamus, and the Central Thalamus. In another embodiment the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers.

Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound-Seattle-31/07-Feb. 8, 2002), typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

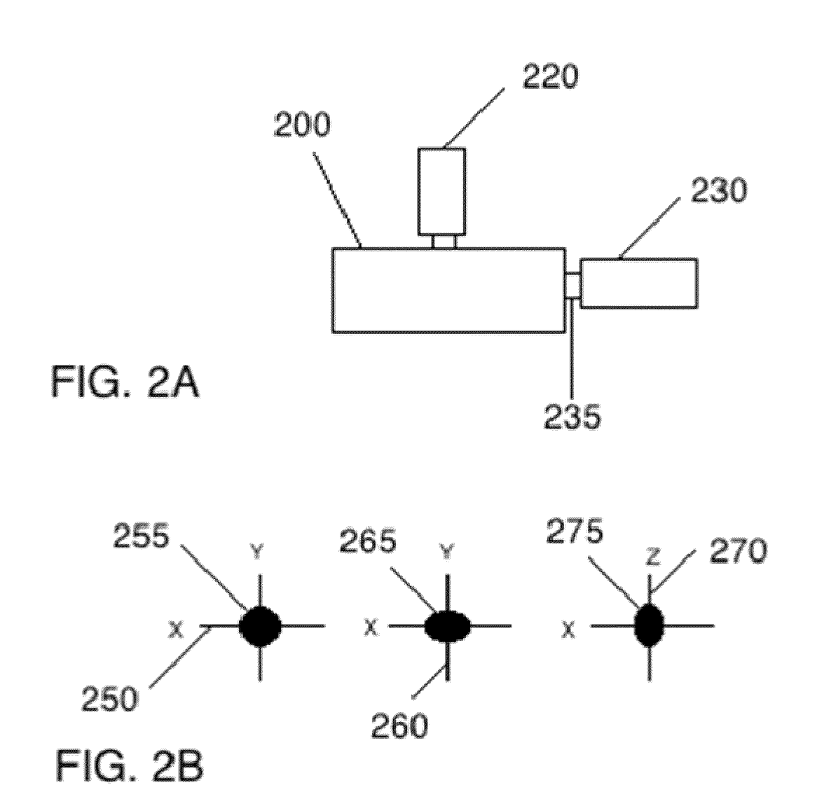

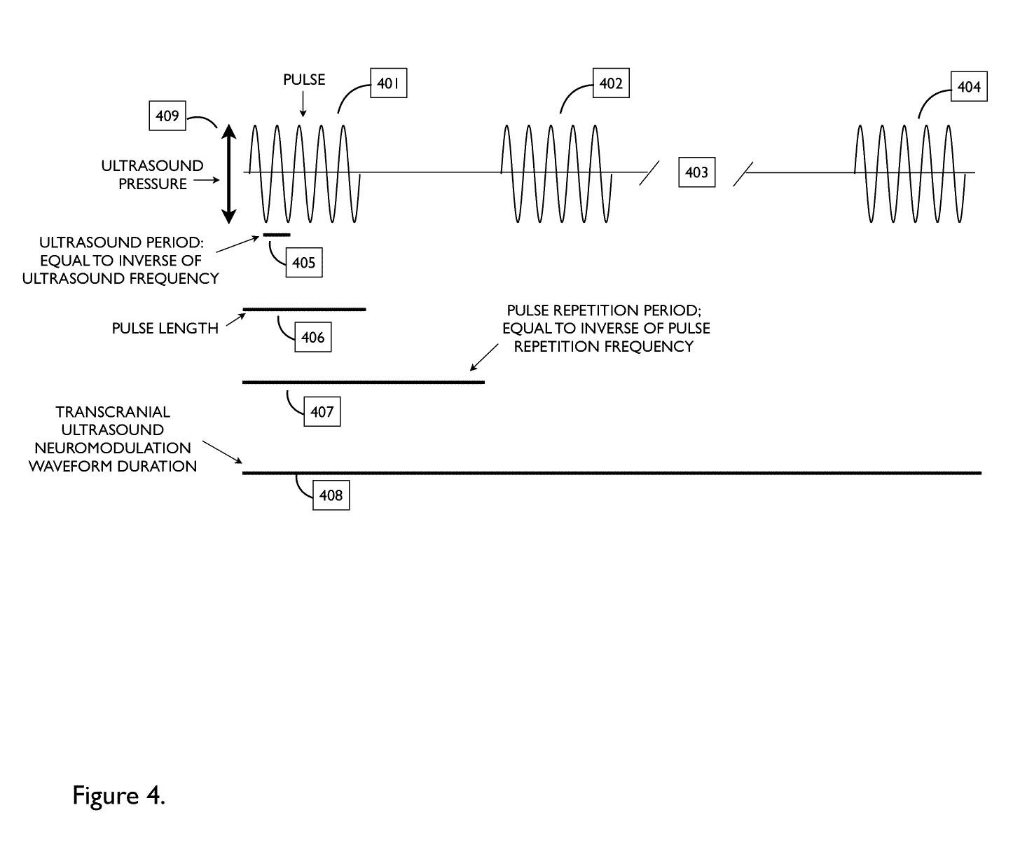

FIGS. 2A and 4B show the mechanism for mechanical perturbation of the ultrasound transducer. In FIG. 2A illustrates a plan view with mechanical actuators 220 and 230 moving ultrasound transducer 200 in and out and left respectively. Actuator rod 235 provides the mechanical interface between mechanical actuator 230 and ultrasound transducer 200 as an example. Not shown is an equivalent mechanical actuator moving ultrasound transducer 200 along an axis perpendicular to the page. Such mechanical actuators can have alternative configurations such as motors, vibrators, solenoids, magnetostrictive, electrorestrictive ceramic and shape memory alloys. Piezo-actuators such as those provided by DSM can have very fine motions of 0.1% length change. FIG. 2B shows effects on the focused ultrasound modulation focused at the target. The axes are 250 (x,y), 260 (x,y,) and 270 (x,z). As demonstrated on 250 the excursions along x and y from 230 and 220 are equal so the resultant pattern is a circle. As demonstrated on 260 the excursion due to 230 is greater than that if 220 so the resultant pattern is longer along the x axis than the y axis. As demonstrated on 470, the excursion is longer along the z axis than the x axis to the resultant pattern is long along the z axis than the x axis. Not shown is the inclusion of the impacts of actuation along the axis perpendicular to the page. In each case, the pattern of movement would be matched to the shape of the target of the modulation.

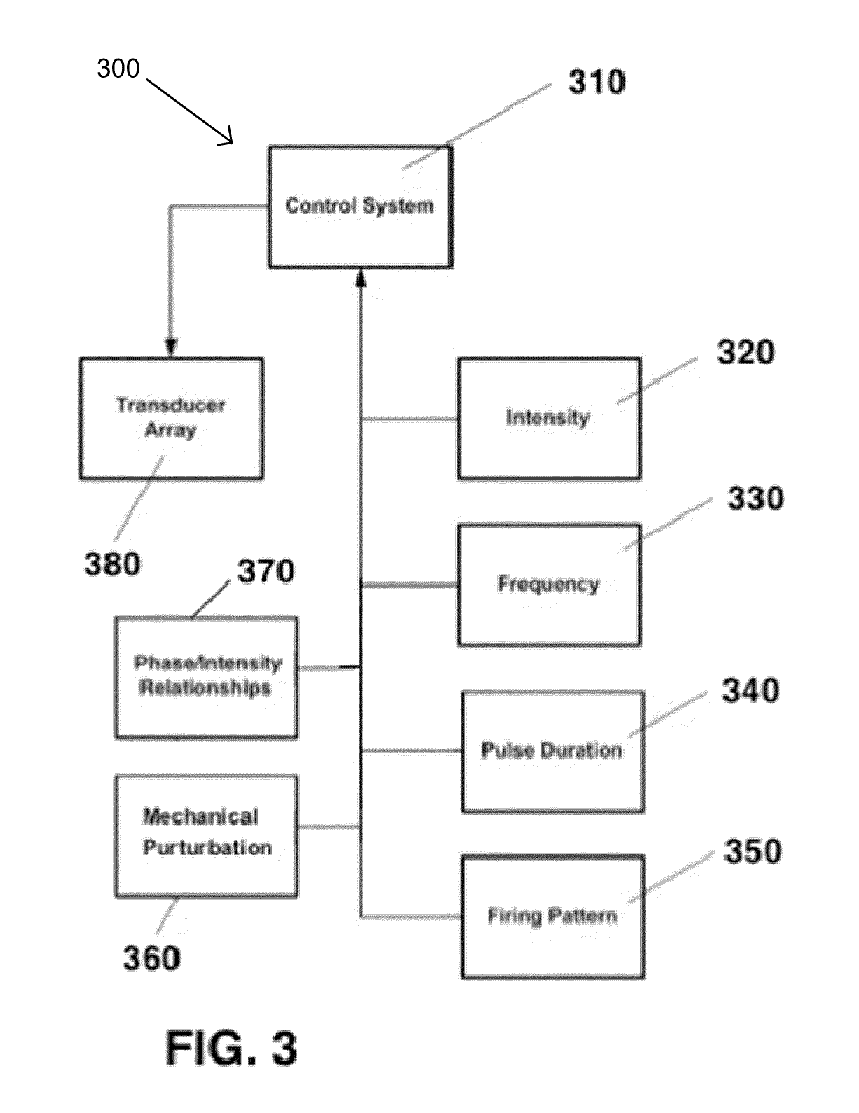

FIG. 3 shows an embodiment of therapeutic ultrasound system 300 comprising a control circuit. The system 300 may comprise a control system 310. The positioning and emission characteristics of transducer array 380 are controlled by control system 310 with control input with neuromodulation characteristics determined by settings of intensity 320, frequency 330, pulse duration 340, firing pattern 350, mechanical perturbation 360, and phase/intensity relationships 370 for beam steering and focusing on neural targets.

The control system 310 may comprise a processor having a computer readable memory embodying instructions of a treatment protocol as described herein. Alternatively or in combination, the control system 310 may comprise programmable array logic (PAL) circuitry embodying instructions of a treatment protocol as described herein. The processor, or PAL, and combinations thereof, can be configured with instructions to provide a treatment in accordance with the methods as described herein. For example, the embodied instructions may provide the configuration for the treatment protocol comprising one or more of the ultrasound frequency within a range as described herein, the pulse length within a range as described herein, a pulse repetition frequency within a range as described herein, or a number of cycles per pulse within a range as described herein, for example.

The patient can be treated in one or more of many ways. For example, the patient can be treated with one or more sessions. The pulse can be shaped in many ways with one or more of macro pulse shaping and amplitude modulation, for example.

In another embodiment, a feedback mechanism to ultrasound neuromodulation is applied such as functional Magnetic Resonance Imaging (fMRI), Positive Emission Tomography (PET) imaging, video-electroencephalogram (V-EEG), acoustic monitoring, thermal monitoring, and patient feedback. In an embodiment, feedback is provided by a measurement specific to a symptom or disease state of a patient.

In still other embodiments, other energy sources are used in combination with or substituted for ultrasound transducers that are selected from the group consisting of Transcranial Magnetic Stimulation (TMS), deep-brain stimulation (DBS), optogenetics application, radiosurgery, Radio-Frequency (RF) therapy, behavioral therapy, and medications.

The embodiments as described herein allow ultrasound stimulation adjustments in variables such as carrier frequency and/or neuromodulation frequency, pulse duration, pulse pattern, mechanical perturbation, as well as the direction of the energy emission, intensity, frequency, duty cycle, phase/intensity relationships to targeting and accomplishing up-regulation and/or down-regulation, dynamic sweeps, and position.

In a preferred embodiment, transcranial ultrasound neuromodulation is used to enhance cognitive function in a subject. One or more ultrasound transducers are coupled to the head of an individual subject such as a human or animal (the ‘recipient’). Components of the transcranial ultrasound neuromodulation device can be near or wearably attached to the recipient in order to provide power and control the intensity, timing, targeting, and waveform characteristics of the transmitted acoustic waves as described herein. A transcranial ultrasound neuromodulation protocol is triggered that uses a waveform. The waveform has an acoustic frequency between about 100 kHz and about 10 MHz and a spatial-peak, temporal-average intensity between about 0.0001 mW/cm² and about 1 W/cm² at the target tissue site (or between 21 mW/cm² and 1 W/cm² in alternative embodiments). The pulsed energy waveform can be configured such that it does not induce heating of the brain due to transcranial ultrasound neuromodulation that exceeds about 2 degrees Celsius for more than about 5 seconds, for example. The transcranial ultrasound neuromodulation protocol induces an effect on neural circuits in one or more brain regions. The effect of transcranial ultrasound neuromodulation on brain function is detected subjectively by the recipient as an improvement in perception, motor control, ideation, decision-making, or cognitive function, or by modifying the recipient's cognitive, emotional, physiological, attentional, or other cognitive state. The effect of the transcranial ultrasound can be determined through physiological measurement of brain activity by one or a plurality of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), or other techniques for measuring brain activity known to one skilled in the art. Alternatively or in combination, the effect of transcranial ultrasound neuromodulation on brain function can be detected with physiological measurement of the body such as by electromyogram (EMG), galvanic skin response (GSR), heart rate, blood pressure, respiration rate, pupil dilation, eye movement, gaze direction, or other physiological measurement.

In an embodiment of the invention, the stimulation frequency for inhibition is lower than 500 Hz (depending on condition and patient). In an embodiment of the invention, the stimulation frequency for excitation is in the range of 500 Hz to 0.25 MHz. In an embodiment of the invention, the ultrasound acoustic frequency is in range of 0.3 MHz to 0.8 MHz with power generally applied not less than 21 mW/cm² but also at higher target- or patient-specific levels at which no tissue damage is caused. In other embodiments of the invention, the ultrasound acoustic frequency is in range of 0.1 MHz to 0.3 MHz. In other embodiments of the invention, the ultrasound acoustic frequency is in range of 0.8 MHz to 10 MHz. In an embodiment of the invention, the lower limit of the spatial-peak temporal-average intensity (Iₛₚₜₐ) of the ultrasound energy at a target tissue site is chosen from the group of: 21 mW/cm², 25 mW/cm², 30 mW/cm², 40 mW/cm², or 50 mW/cm². In an embodiment of the invention, the upper limit of the Iₛₚₜₐ of the ultrasound energy at a target tissue site is chosen from the group of: 1000 mW/cm², 500 mW/cm², 300 mW/cm², 200 mW/cm², 100 mW/cm², 75 mW/cm², or 50 mW/cm².

In an embodiment of the invention, the acoustic frequency is modulated at the lower rate to impact the neuronal structures as desired (e.g., say typically 300 Hz for inhibition (down-regulation) or 1 kHz for excitation (up-regulation). The modulation frequency (superimposed on the carrier frequency of say 0.5 MHz or similar) may be divided into pulses 0.1 to 20 msec. In an embodiment of the invention, the pulses are repeated at frequencies of 2 Hz or lower for down regulation and higher than 2 Hz for up regulation although this will be both patient and condition specific. The number of ultrasound transducers can vary between one and 500 hundred. In an embodiment of the invention, ultrasound therapy can be combined with therapy using other neuromodulation modalities, such as one or more of Transcranial Magnetic Stimulation (TMS) or transcranial Direct Current Stimulation (tDCS), for example.

A transcranial ultrasound neuromodulation protocol delivers ultrasound to one or more brain regions and induces neuromodulation that correlates more strongly in time with the time course of mechanical effects on tissue than thermal effects. The acoustic frequency for transcranial ultrasound neuromodulation is generally greater than about 100 kHz and less than about 10 MHz (205, 303, FIGS. 2 and 3), i.e. generally greater than about 100 kHz and less than about 10 MHz; optionally greater than about 0.3 MHz and less than about 0.8 MHz; optionally greater than about 0.3 MHz and less than about 1 MHz; optionally greater than about 0.3 MHz and less than about 0.5 MHz; optionally greater than about 0.3 MHz and less than about 0.4 MHz; optionally greater than about 0.3 MHz and less than about 0.6 MHz; optionally greater than about 0.3 MHz and less than about 10 MHz; optionally greater than about 0.25 MHz and less than about 0.8 MHz; optionally greater than about 0.25 MHz and less than about 1 MHz; optionally greater than about 0.25 MHz and less than about 0.5 MHz; optionally greater than about 0.25 MHz and less than about 0.4 MHz; optionally greater than about 0.25 MHz and less than about 0.6 MHz; optionally greater than about 0.25 MHz and less than about 10 MHz; optionally greater than about 0.1 MHz and less than about 0.8 MHz; optionally greater than about 0.1 MHz and less than about 1 MHz; optionally greater than about 0.1 MHz and less than about 0.5 MHz; optionally greater than about 0.1 MHz and less than about 0.4 MHz; optionally greater than about 0.1 MHz and less than about 0.6 MHz; optionally greater than about 0.1 MHz and less than about 10 MHz; optionally greater than about 0.5 MHz and less than about 0.8 MHz; optionally greater than about 0.5 MHz and less than about 1 MHz; optionally greater than about 0.5 MHz and less than about 0.55 MHz; optionally greater than about 0.5 MHz and less than about 0.7 MHz; optionally greater than about 0.5 MHz and less than about 0.6 MHz; optionally greater than about 0.5 MHz and less than about 10 MHz; optionally greater than about 0.7 MHz and less than about 0.8 MHz; optionally greater than about 0.7 MHz and less than about 1 MHz; optionally greater than about 0.7 MHz and less than about 0.75 MHz; or optionally greater than about 0.5 MHz and less than about 10 MHz. Particularly advantageous acoustic frequencies are between about 0.3 MHz and about 0.7 MHz. The spatial-peak temporal-average (Iₛₚₜₐ) intensity of the ultrasound wave in brain tissue is greater than about 0.0001 mW/cm² and less than about 1 W/cm², i.e. generally from 21 mW/cm² to 0.1 W/cm2; optionally from 21 mW/cm² to 0.5 W/cm²; optionally from 21 mW/cm² to 1 W/cm²; optionally from 50 mW/cm² to 0.1 W/cm²; optionally from 50 mW/cm² to 0.5 W/cm2; optionally from 50 mW/cm² to 1 W/cm²; optionally from 0.1 W/cm² to 0.2 W/cm²; optionally from 0.1 W/cm² to 0.5 W/cm²; and optionally from 0.1 W/cm² to 1 W/cm². Particularly advantageous Iₛₚₜₐ values are between about 100 mW/cm² and about 700 mW/cm², usually in the range from about 200 mW/cm² to about 500 mW/cm². The Iₛₚₜₐ value for any particular transcranial ultrasound neuromodulation protocol is calculated according to methods well known in the art that relate to the ultrasound pressure and temporal average of the transcranial ultrasound neuromodulation waveform over its duration. Effective ultrasound intensities for activating neurons or neuronal circuits do not cause tissue heating greater than about 2 degrees Celsius, usually less than 1 degree Celsius, for a period longer than about 5 seconds, preferably no longer than 3 seconds.

Substantial attenuation of ultrasound intensity occurs at the boundaries between skin, skull, dura, and brain due to impedance mismatches, absorption, and reflection so the required ultrasound intensity delivered to the skin or skull may exceed the intensity at the targeted brain region by up to 10-fold or more depending on skull thickness and other tissue and anatomical properties, and a person of ordinary skill in the art can adjust the intensity and frequencies as described herein so as to provide appropriate amounts of ultrasound energy to the target tissue.

FIG. 4 shows transcranial ultrasound neuromodulation waveform and pulsed ultrasound protocol.

Ultrasound delivered in a plurality of pulses 401, 402, 404 is an effective configuration for activating neurons that reduces the temporal average intensity while also achieving desired brain stimulation or neuromodulation effects for a transcranial ultrasound neuromodulation waveform of a particular duration 408. In addition to acoustic frequency 405 and transducer variables, several waveform characteristics such as cycles per pulse, pulse repetition period 407, number of pulses, and pulse length affect the intensity characteristics and outcome of any particular transcranial ultrasound neuromodulation stimulus on brain activity.

A pulsed transcranial ultrasound neuromodulation protocol generally uses pulse lengths 406 within a range from about 0.5 microseconds to about 1 second, i.e. generally from 0.5 microseconds to 5 microseconds; optionally from 0.5 microseconds to 50 microseconds; optionally from 0.5 microseconds to 100 microseconds; optionally from 0.5 microseconds to 500 microseconds; optionally from 0.5 microseconds to 1 ms; optionally from 0.5 microseconds to 10 ms; optionally from 0.5 microseconds to 100 ms; optionally from 0.5 microseconds to 500 ms; optionally from 0.5 microseconds to 1 second; optionally from 5 microseconds to 50 microseconds; optionally from 5 microseconds to 100 microseconds; optionally from 5 microseconds to 500 microseconds; optionally from 5 microseconds to 1 ms; optionally from 5 microseconds to 10 ms; optionally from 5 microseconds to 100 ms; optionally from 5 microseconds to 500 ms; optionally from 5 microseconds to 1 second; optionally from 100 microseconds to 500 microseconds; optionally from 100 microseconds to 1 ms; optionally from 100 microseconds to 10 ms; optionally from 100 microseconds to 100 ms; optionally from 100 microseconds to 500 ms; optionally from 100 microseconds to 1 second; optionally from 500 microseconds to 1 ms; optionally from 500 microseconds to 10 ms; optionally from 500 microseconds to 100 ms; optionally from 500 microseconds to 500 ms; optionally from 500 microseconds to 1 second; optionally from 1 ms to 10 ms; optionally from 1 ms to 100 ms; optionally from 1 ms to 500 ms; optionally from 1 ms to 1 second; and optionally from and 100 ms to 1 second, for example.

A transcranial ultrasound neuromodulation protocol may use one or more pulse repetition frequencies 407 (PRFs) within a range from about 50 Hz to about 25 kHz, i.e. generally from 50 Hz to 100 Hz; optionally from 50 Hz to 250 Hz; optionally from 50 Hz to 1 kHz; optionally from 50 Hz to 2 kHz; optionally from 50 Hz to 3 kHz; optionally from 50 Hz to 4 kHz; optionally from 50 Hz to 5 kHz; optionally from 50 Hz to 10 kHz; optionally from 50 Hz to 25 kHz; optionally from 100 Hz to 250 Hz; optionally from 100 Hz to 1 kHz; optionally from 100 Hz to 2 kHz; optionally from 100 Hz to 3 kHz; optionally from 100 Hz to 4 kHz; optionally from 100 Hz to 5 kHz; optionally from 100 Hz to 10 kHz; optionally from 100 Hz to 25 kHz; optionally from 250 Hz to 500 Hz; optionally from 250 Hz to 1 kHz; optionally from 250 Hz to 2 kHz; optionally from 250 Hz to 3 kHz; optionally from 250 Hz to 4 kHz; optionally from 250 Hz to 5 kHz; optionally from 250 Hz to 10 kHz; optionally from 250 Hz to 25 kHz; optionally from 500 Hz to 1 kHz; optionally from 500 Hz to 2 kHz; optionally from 500 Hz to 3 kHz; optionally from 500 Hz to 4 kHz; optionally from 500 Hz to 5 kHz; optionally from 500 Hz to 10 kHz; optionally from 500 Hz to 25 kHz; optionally from 1 kHz to 2 kHz; optionally from 1 kHz to 3 kHz; optionally from 1 kHz to 4 kHz; optionally from 1 kHz to 5 kHz; optionally from 1 kHz to 10 kHz; optionally from 1 kHz to 25 kHz; optionally from 3 kHz to 4 kHz; optionally from 3 kHz to 5 kHz; optionally from 3 kHz to 10 kHz; optionally from 3 kHz to 25 kHz; optionally from 5 kHz to 10 kHz; optionally from 5 kHz to 25 kHz; and optionally from and 10 kHz to 25 kHz. Particularly advantageous PRFs are generally between about 1 kHz and about 3 kHz, for example.

For pulsed transcranial ultrasound neuromodulation waveforms, the number of cycles per pulse (cpp) can be within a range from about 5 to about 10,000,000, for example. Particularly advantageous cpp values vary depending on the choice of other transcranial ultrasound neuromodulation parameters and can be generally between about 10 and about 250, for example. The number of pulses for pulsed transcranial ultrasound neuromodulation waveforms can be between about 1 pulse and about 125,000 pulses, for example. Particularly advantageous pulse numbers for pulsed transcranial ultrasound neuromodulation waveforms can be between about 100 pulses and about 250 pulses, for example.

The pulses may comprise a duty cycle configured to activate neurons with decreased heating. The pulse length 406 and pulse repetition period 407 correspond to the duty cycle, and in many embodiments the duty cycle encompasses the ratio of the pulse length to the pulse repetition period multiplied by one hundred. The duty cycle can be less than 50%, and can be within a range from about 0.1% to about 50%, for example within a range from about 1% to 25%. In an embodiment, the ultrasound frequency can be about 0.5 MHz, the pulse duration about 20 microseconds, and the pulse repetition period about 2000 microseconds, such that the duty cycle comprises about 1%, for example. Based on the teachings described herein a person of ordinary skill in the art can configure the duty cycle in many ways, and the duty cycle can be less than 50%, less than 25%, less than 10% and less than 5%, for example.

In many embodiments, the configuration for the treatment protocol comprises one or more of the ultrasound frequency within a range as described herein, the pulse length within a range as described herein, a pulse repetition frequency within a range as described herein, a number of cycles per pulse within a range as described herein, or a duty cycle as described herein, for example.

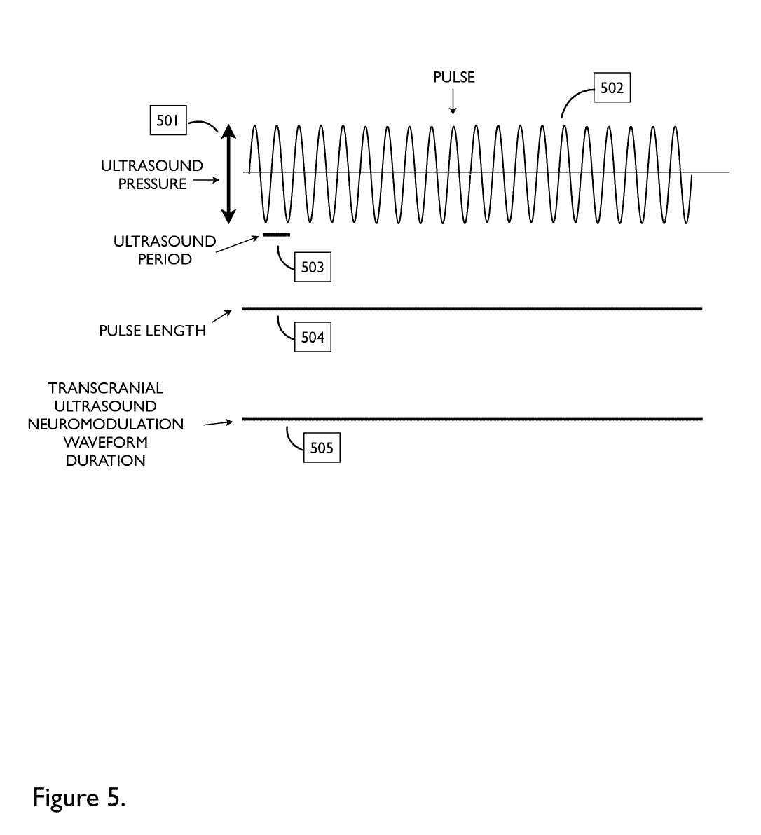

FIG. 5 shows transcranial ultrasound neuromodulation waveform and continuous wave ultrasound protocol.

Tone bursts 502 that extend for about 1 second or longer can be referred to as continuous wave (CW), although the tone bursts 502 may comprise pulses, for example. In alternative embodiments, a transcranial ultrasound neuromodulation waveform comprises one or more continuous wave (CW) ultrasound waveforms less than about five seconds in duration 505, typically with a CW pulse length 504 being from 1 second to 5 seconds. US protocols that include such CW waveforms offer advantages for neuromodulation due to their capacity to drive activity robustly. However, one potential disadvantage of transcranial ultrasound neuromodulation protocols with CW pulses is that the temporal average intensity can be higher which may cause painful thermal stimuli on the scalp or skull and may also induce heating and thus damage in brain tissue. Thus, advantageous embodiments using CW pulses may employ a lower acoustic intensity 501 and/or a slow pulse repetition frequency of less than about 1 Hz, as can be determined by a person of ordinary skill in the art based on the embodiments described herein. For instance, a CW US stimulus waveform with 1 second pulse lengths repeated at 0.5 Hz would deliver US every other second. Alternative pulsing protocols including those with slower pulse repetition frequencies of less than about 0.5 Hz or less than about 0.1 Hz or less than about 0.01 Hz or less than about 0.001 Hz are also beneficial. In some useful embodiments, the interval between pulses or pulse length may be varied during a transcranial ultrasound neuromodulation protocol that include CW pulses.

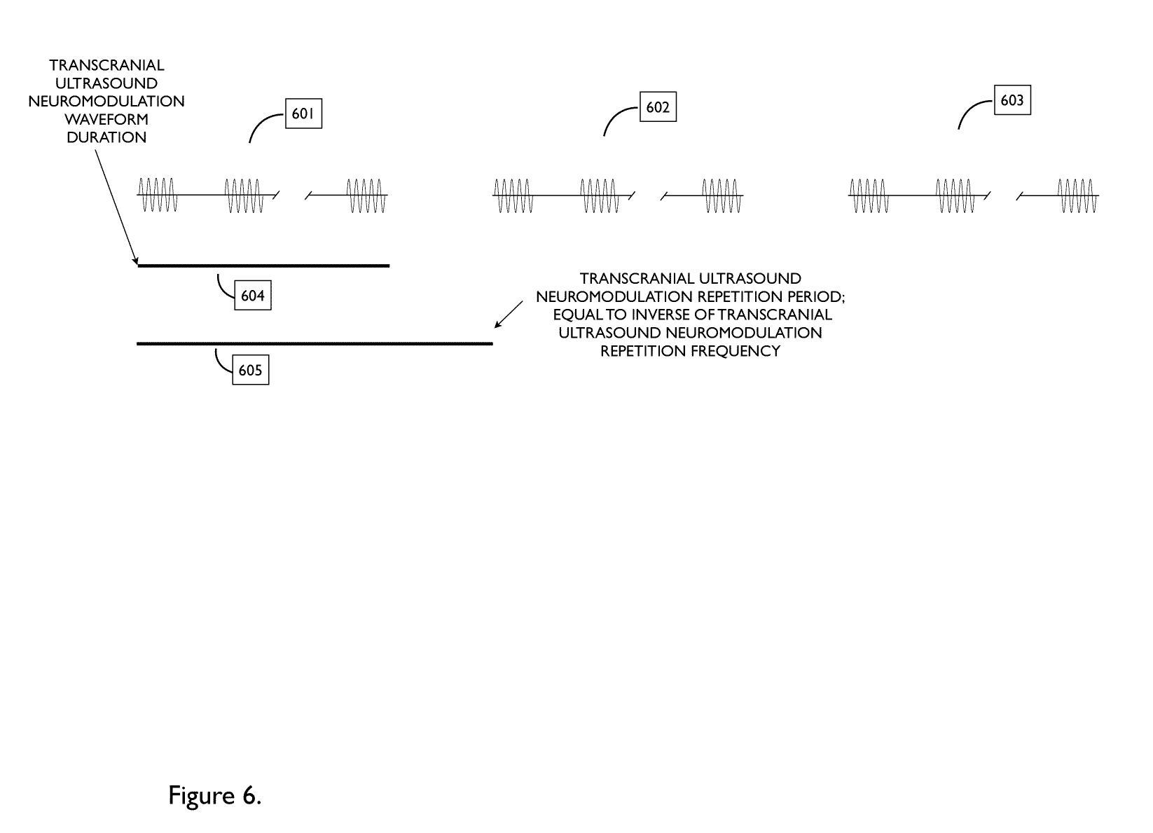

FIG. 6 shows transcranial ultrasound neuromodulation waveform repetition.

In some embodiments, repeating the transcranial ultrasound neuromodulation protocol 601, 602, 603 is advantageous for achieving particular forms of neuromodulation to enhance or modify cognitive function. In some embodiments, the number of times a transcranial ultrasound neuromodulation protocol of appropriate duration 604 is repeated is chosen to be in the range between 2 times and 100,000 times. Particularly advantageous numbers of transcranial ultrasound neuromodulation protocol repeats can be between 2 and 1,000 repeats, for example. The repetition frequency of a transcranial ultrasound neuromodulation protocol 605 may be less than about 10 Hz, less than about 1 Hz, less than about 0.1 Hz, or lower, for example. The transcranial ultrasound neuromodulation repetition frequency may be fixed or variable. Variable transcranial ultrasound neuromodulation repetition frequency values may be random, pseudo-random, ramped, or otherwise modulated. The transcranial ultrasound neuromodulation repetition period is defined as the inverse of the transcranial ultrasound neuromodulation repetition frequency.

Providing a combination of ultrasound frequencies is useful for efficient brain stimulation. Various configurations for achieving a combination of ultrasound frequencies to the brain of the user can be determined. A configuration for producing ultrasound waves that contain power in a range of frequencies is to use square waves to drive the transducer. Another configuration for generating a mixture of ultrasound frequencies is to choose transducers that have different center frequencies and drive each at their resonant frequency. One or more of the above configurations or alternative configurations known to those skilled in the art for generating US waves with a combinations of frequencies may be combined in accordance with embodiments described herein. Mixing, amplitude modulation, or other configurations for generating more complex transcranial ultrasound neuromodulation waveforms can be beneficial for driving distinct brain wave activity patterns or to bias the power, phase, or spatial extent of brain oscillations such as slow-wave, delta, beta, theta, gamma, or alpha rhythms, for example.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 MHz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

The ultrasound neuromodulation can be administered in sessions. Examples of session types include periodic sessions, such as a single session of length in the range from 15 to 60 minutes repeated daily or five days per week for one to six weeks. Other lengths of session or number of weeks of neuromodulation are applicable, such as session lengths from 1 minute up to 2.5 hours and number of weeks ranging from one to eight. Sessions occurring in a compressed time period typically means a single session of length in the range from 30 to 60 minutes repeated during with inter-session times of 15 minutes to 60 minutes over one to three days. Other inter-session times in the range between 1 minute and three hours and days of compressed therapy such as one to five days are applicable. In an embodiment of the invention, sessions occur only during waking hours. Maintenance consists of periodic sessions at fixed intervals or on as-needed basis such as occurs periodically for tune-ups. Maintenance categories are maintenance post-completion of original treatment at fixed intervals and maintenance post-completion of original treatment with as-needed maintenance tune-ups as defined by a clinically relevant measurement. In an embodiment that uses fixed intervals to determine when additional ultrasound neuromodulation sessions are delivered, one or more 50-minute sessions occur during the second week the 4^{th} and 8^{th} months following the first treatment. In an embodiment that when additional ultrasound neuromodulation sessions are delivered based on a clinically-relevant measurement, one or more 50-minute sessions occur during week 7 because a tune up is needed at that time as indicated by the re-emergence of symptoms. Use of sessions is important for the retraining of neural pathways for change of function, maintenance of function, or restoration of function. Retraining over time, with intermittent reinforcement, can more effectively achieve desired impacts. Efficient schedules for sessions are advantageous so that patients can minimize the amount of time required for their ultrasound treatments. Such neuromodulation systems can produce applicable acute or long-term effects. The latter occur through Long-Term Depression (LTD) or Long-Term Potentiation (LTP) via training.

Work in relation to embodiments as described herein suggests that differences in FUP phase, frequency, and amplitude produce different neural effects. Low frequencies (defined as below 500 Hz.) can be inhibitory in at least some embodiments. High frequencies (in the range of 500 Hz to 5 MHz, for example from 500 Hz to 0.25 MHz) can be excitatory and activate neural circuits in at least some embodiments. In many embodiments, this targeted inhibition or excitation based on frequency works for the targeted region comprising one or more of gray or white matter. Repeated sessions may result in long-term effects. The cap and transducers to be employed can be preferably made of non-ferrous material to reduce image distortion in fMRI imaging, for example. In many embodiments, if after treatment the reactivity as judged with fMRI of the patient with a given condition becomes more like that of a normal patient, this clinical assessment may be indicative of treatment effectiveness. In many embodiments, the FUP is to be applied 1 ms to 1 s before or after the imaging. Alternatively or in combination, a CT (Computed Tomography) scan can be run to gauge the bone density and structure of the skull, which can be used to determine one or more of the carrier wave frequency, the pulse intensity, the pulse energy, the pulse duration, the pulse repetition rate, or the pulse phase, for a series of pulses as described herein, for example.

In many embodiments focused ultrasound pulses (FUP) are produced by multiple ultrasound transducers, preferably in the range of 300 to 1000, arranged in a cap place over the skull to affect a multi-beam output. These transducers can be coordinated by a computer and used in conjunction with an imaging system, preferable an fMRI (functional Magnetic Resonance Imaging), but possibly a PET (Positron Emission Tomography) or V-EEG (Video-Electroencephalography) device. The user can interact with the computer to direct the FUP to the desired point in the brain, sees where the stimulation actually occurred by viewing the imaging result, and thus adjusts the position of the FUP accordingly. The position of focus can be obtained by adjusting the phases and amplitudes of the ultrasound transducers in accordance with methods known to one of ordinary skill in the art. The imaging may also illustrate the functional connectivity of the target and surrounding neural structures. The focus can be two or more centimeters deep and 0.5 to 1000 mm in diameter, or preferably in the range of 2-12 cm deep and 0.5-2 mm in diameter, for example. Either a single FUP or multiple FUPs can be applied to either one or multiple live neuronal circuits. Differences in FUP phase, frequency, and amplitude can produce different neural effects. Low frequencies (defined as below 500 Hz.) can be inhibitory. High frequencies (in the range of 500 Hz to 5 MHz, for example from 500 Hz to 0.25 MHz) can be excitatory and activate neural circuits. This works whether the target is gray or white matter. Repeated sessions result in long-term effects. The cap and transducers to be employed are preferably made of non-ferrous material to reduce image distortion in fMRI imaging. After treatment the reactivity as judged with fMRI of the patient with a given condition can become more like that of a normal patient and indicative of treatment effectiveness. The FUP can be applied 1 ms to 1 s before or after the imaging. In addition a CT (Computed Tomography) scan can be run to gauge the bone density and structure of the skull.

In specific embodiments, the acoustic energy can be directed at/or to a target region in the brain to cause a selected cognitive effect. Specific embodiments of target regions and cognitive effects suitable for combination in accordance with the embodiments described herein may comprise one or more combinations from each row of Table 1.

TABLE 1

| Cognitive effect | Target region |
|---|---|
| Perception of touch | Somatosensory cortex |
| Auditory perception | Auditory cortex |
| Vestibular | Temporal-parietal junction, central sulcus, |
| perception | intraparietal sulcus, and insular cortex |
| Visual perception | Primary and extrastriate visual cortex |
| Olfactory perception | Piriform cortex |
| Language | Wernicke's area |
| comprehension | |
| Language | Broca's area |
| production | |
| Long-term memory | Hippocampus and parahippocampal formation (and |
| | connected portions of cortex, e.g. entorhinal cortex |
| | and perirhinal cortex) |
| Modulation of pain | Rostral anterior cingulate cortex |
| processing | |
| Emotion | Limbic system (e.g. amygdala) |
| Motor control and | Primary and supplementary motor cortex; thalamus; |
| movements | cerebellum; basal ganglia; substantia nigra |
| Attention | Gamma rhythms |
| Relaxation | Alpha rhythms |
| Empathy, social | Brainstem nuclei, hypothalamus, amygdala, anterior |
| interaction | cingulated cortex, prefrontal cortex, ventromedial |
| | prefrontal cortex, and other brain regions involved |
| | in oxytocin and arginine vasopressin function |
| Mirth and laughter | Inferior temporal gyms, cingulated gyms, |
| | subthalamic nucleus |
| Fear | Amygdala, insular cortex, internal capsule, nucleus |
| | accumbens, and anterior temporal gyms |
| Physiological | Various brainstem nuclei |
| arousal, sleep state | |
| Modulation of risk | Dorsolateral prefrontal cortex |
| taking | |

Table 1 lists cognitive effects in one column and target regions in another column, and the specific combinations are contained within each row of the table. For example, the cognitive effect of touch perception can be enhanced by treatment of the target region comprising somatosensory cortex. A person of ordinary skill in the art can determine appropriate treatment parameters such as one or more of the ultrasound frequency within a range as described herein, the pulse length within a range as described herein, a pulse repetition frequency within a range as described herein, a number of cycles per pulse within a range as described herein, or a duty cycle as described herein, for example, so as to treat the target region corresponding to the cognitive effect as set forth in a row of Table 1 with decreased amounts of energy and increased cognitive effect in a repeatable and reliable manner. Additional treatment parameters can be determined for each cognitive effect and target location in each row of Table 1. For example, optimal treatment parameters for the cognitive effect comprising the modulation of risk taking based on the treatment region comprising the Dorsolateral prefrontal cortex can be determined. These treatment parameters can be stored on the computer readable memory of the system controller as describe herein.

Based on the embodiments and teachings disclosed herein, a person of ordinary skill in the art can conduct clinical investigations on human subjects to determine appropriate treatment protocols comprising ultrasound parameters as described herein in order to determine appropriate treatment regions and cognitive effects. The measured output in response to the ultrasound treatment may comprise electroencephalography (EEG), positron emission tomography, magnetic resonance imaging, or other known imaging to determine the effect of the ultrasound parameters. Subjective measurements may also be used such as known cognition test to determine parameters suitable for increasing cognition.

The following publications are provided as enabling description that can be combined with the teachings as described herein by a person of ordinary skill in the art in order to practice embodiments as described herein without undue experimentation.

The stimulation of deep-brain structures with ultrasound has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton describes a strong DC magnetic field as well and describes the mechanism as that given that the tissue to be stimulated is conductive that particle motion induced by an ultrasonic wave will induce an electric current density generated by Lorentz forces, such that ultrasound is suitable for combination with TMS in accordance with embodiments as described herein.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as compared to TMS focused to no more than 1 cm. However, a person of ordinary skill in the art can combine ultrasound with TMS in accordance with the embodiments as described herein.

Deisseroth and Schneider (U.S. patent application Ser. No. 12/263,026 published as US 2009/0112133 A1, Apr. 30, 2009) describe an alternative approach suitable for combination in accordance with embodiments described herein, with modifications of neural transmission patterns between neural structures and/or regions are described using ultrasound (including use of a curved transducer and a lens) or RF. The impact of long-term potentiation (LTP) and long-term depression (LTD) for durable effects can be achieved in accordance with embodiments described herein.

Neural targets identified include the Ventral Tegmentum, the Hypothalamus, the Central Thalamus (Shirvalkar, P., Seth, M., Schiff, N. D., and D. G. Herrera, “Cognitive Enhancement with Central Thalamic Electrical Stimulation,” PNAS Nov. 7, 2006 vol. 103 no. 45 17007-17012), the anterior cortex, and the Fronto-Temporal Lobe. Lazano and Mayberg (U.S. Patent Application 2006/0201090, “Method of Treating Cognitive Disorders Using Neuromodulation”) describe an invention using electrical and/or chemical stimulation of a variety of targets for the treatment of a variety of conditions but are non-specific about what target is related to what condition and do not cover cognitive enhancement in normal individuals.

Snyder and his colleagues have studied the impact of TMS used to inhibit anterior areas (including the Fronto-Temporal Lobe) of the brain on normal subjects (Snyder, A., Bossomaier, T., and D. J. Mitchell, “Concept Formation: ‘Object’ Attributes Dynamically Inhibited from Conscious Awareness,” Journal of Integrative Neuroscience 3(1), 31-46, 2004 and Snyder, A. W., Mulcahy, E., J. L., Taylor, et al., “Savant-Like Skills Exposed in Normal People by Suppressing the Left Fronto-Temporal lobe. Journal of Integrative Neuroscience 2(2), 149-158, 2003). They found that both ability to spell check was improved and that drawing style was changed to a more concrete style. They postulated this was due to reducing top-down semantic control. This could be related to work of Miller et al. (Miller, B. L., Ponton, M., Benson, D. F., Cummings, J. L., & I. Mena, “Enhanced artistic creativity with temporal lobe degeneration,” Lancet, 348, 1744-1755, 1996) who looked at previously normal patients with Fronto-Temporal Lobe Dementia who demonstrated emergence of new artistic skills along with their dementia, although attributing this to a different neural mechanism. Miller and colleagues attributed this to deterioration of the Orbito-Frontal Lobe and Anterior Temporal Lobe resulting in an impact on visual systems related to perception whose inhibition was decreased.

With respect to calendar calculation, Boddaert et al. (Boddaert, N., Barthelemy, C., Poline, J. B., Samson, Y., Brunelle, F., & M. Zilbovicius, M., “Autism: Functional brain mapping of exceptional calendar capacity,” British Journal of Psychiatry, 187, 83-86, 2005) used PET imaging compared calendar calculation to rest in an adult with autism. This demonstrated activation of brain regions usually associated with memory (Left Hippocampus, Left Frontal Cortex, and Left Middle Temporal Lobe).

Bystritsky (U.S. Pat. No. 7,283,861) describes concurrent imaging suitable for incorporation in accordance with embodiments.

Clement and Hynynen described the position of focus obtained by adjusting the phases and amplitudes of ultrasound transducers suitable for combination in accordance with embodiments described herein (“A non-invasive method for focusing ultrasound through the human skull,” Phys. Med. Biol. 47 (2002) 1219-1236).

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of deep-brain neuromodulation of a subject with ultrasound stimulation, the method comprising:
focusing one or more ultrasound transducers at one or more neural targets related to cognitive enhancement;
applying pulsed power to the one or more ultrasound transducers to transmit ultrasound to the one or more neural targets; and
controlling the pulsed power to enhance a cognitive function of the subject.

2. The method of claim 1, wherein the one or more neural targets comprises one or more of a hippocampus, a cingulate cortex, a thalamus, a hypothalamus, a cerebellum, an amygdala, or a nucleus accumbens and wherein an acoustic ultrasound frequency is in a range from 0.3 MHz to 0.8 MHz.

3. The method of claim 1, wherein:
a. the ultrasound is transmitted into a brain of the subject with the one or more ultrasound transducers targeting one or more brain regions related to an emotional state or a mood disorder;
b. an acoustic frequency of the ultrasound is greater than about 100 kHz and less than about 10 MHz;
c. a spatial-peak temporal-average (Iₛₚₜₐ) intensity of an ultrasound waveform at a site of cells of the neural target to be modulated is less than about 1 W/cm²;
d. an ultrasound pulse length is less than about 5 seconds; and
e. the ultrasound induces an effect in the one or more brain regions comprising one or more of neuromodulation, brain activation, neuronal activation, neuronal inhibition, or a change in blood flow, and
f. the one or more neural targets is heated by no more than approximately 2 degrees Celsius for a period greater than about 5 seconds.

4. The method of claim 1, wherein the neural target comprises one or more of,
a Somatosensory cortex, an Auditory cortex, a Temporal-parietal junction, a central sulcus, an intraparietal sulcus, an insular cortex, a primary visual cortex, an extrastriate visual cortex, a Piriform cortex, a Wernicke's area, a Broca's area, a Hippocampus, a parahippocampal formation, an entorhinal cortex, a perirhinal cortex, a Rostral anterior cingulate cortex, a Limbic system, an amygdale, a primary motor cortex, a supplementary motor cortex, a thalamus, a cerebellum, a basal ganglia, a substantia nigra, gamma rhythms, alpha rhythms, Brainstem nuclei, a hypothalamus, an amygdala, an anterior cingulated cortex, a prefrontal cortex, a ventromedial prefrontal cortex, a brain region involved in oxytocin and arginine vasopressin function, an inferior temporal gyms, a cingulated gyms, a subthalamic nucleus, an Amygdala, an insular cortex, an internal capsule, a nucleus accumbens, an anterior temporal gyms, brainstem nuclei, or Dorsolateral prefrontal cortex,
and wherein the cognitive function comprises one or more of
perception of touch, auditory perception, vestibular perception, visual perception, olfactory perception, language comprehension, language production, long-term memory, modulation of pain processing, emotion, motor control and movements, attention, relaxation, empathy, social interaction, mirth, laughter, fear, physiological arousal, sleep state, or modulation of risk taking.

5. The method of claim 1 wherein the one or more neural targets responds to the ultrasound with one or more of an acute response, long-term potentiation, long-term depression, up-regulation, or down-regulation.

6. The method of claim 1 wherein the treated cognitive function has been clinically diagnosed as abnormal and the subject has been diagnosed with a conditioned selected from the group consisting of: Alzheimer's Disease, Parkinson's disease, Creutzfeld-Jacob disease, Attention Deficit Hyperactivity Disorder, dementia, and stroke.

7. The method of claim 1 wherein the ultrasound improves learning by a student studying for a test.

8. The method of claim 1 wherein the one or more neural targets is selected from the group consisting of Orbito-Frontal Cortex (OFC), Anterior Temporal Lobe, Left Hippocampus, Left Frontal Cortex, Left Middle Temporal Lobe, Ventral Tegmentum, Hypothalamus, and the Central Thalamus.

9. The method of claim 1 wherein acoustic energy is delivered by the one or more ultrasonic transducers such that beams intersect at one or more brain targets.

10. The method of claim 1, wherein acoustic energy of the ultrasound has a frequency in a range between 100 kHz and 10 MHz.

11. The method of claim 1 wherein a power of the ultrasound applied is at least 21 mW/cm².

12. The method of claim 1 wherein the power applied is greater than 21 mW/cm² and less than a damage threshold of the one or more neural targets.

13. The method of claim 1 wherein a stimulation frequency of lower than 500 Hz is applied for inhibition of neural activity.

14. The method of claim 12 wherein a modulation frequency of lower than 500 Hz is divided into pulses having a duration within a range from 0.1 to 20 msec. repeated at frequencies of 2 Hz or lower for down regulation.

15. The method of claim 1 wherein a stimulation frequency for excitation is in the range of 500 Hz to 0.25 MHz.

16. The method of claim 14 wherein a modulation frequency of 500 Hz or higher is divided into a plurality of pulses, each pulse of the plurality having a duration of 0.1 to 20 msec. and wherein the plurality of pulses is repeated at frequencies higher than 2 Hz for up regulation.

17. The method of claim 1, wherein a pulse length is within a range between 0.5 microsecond and 5 seconds.

18. The method of claim 1, wherein a pulse repetition frequency is within a range between 50 Hz and 25 kHz.

19. The method of claim 1 wherein mechanical perturbations are applied radially or axially to move the one or more ultrasound transducers.

20. The method of claim 1 wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, and thermal monitoring.

21. The method of claim 1 wherein deep-brain neuromodulation is combined with one or more therapies selected from the group consisting of Transcranial Magnetic Stimulation (TMS), deep-brain stimulation (DBS), application of optogenetics, radiosurgery, Radio-Frequency (RF) therapy, behavioral therapy, and medications.

22. A system for deep-brain neuromodulation of a subject with ultrasound stimulation, the system comprising:
one or more ultrasound transducers configured to focus at one or more neural targets related to cognitive enhancement; and
circuitry configured to apply pulsed power to the one or more ultrasound transducers and control the pulsed power to enhance a cognitive function of the subject.
